SEQUENCE LISTING (21 sequences)

SEQ ID NO: 1 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 2 - protein, 579 aa, Homo sapiens
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 3 - protein, 619 aa, Homo sapiens
MASGAYNPYIEIIEQPRQRGMRFRYKCEGRSAGSIPGEHSTDNNRTYPSIQIMNYYGKGKVRITLVTKNDPYKPHPHDLVGKDCRDGYYEAEFGQERRPLFFQNLGIRCVKKKEVKEAIITRIKAGINPFNVPEKQLNDIEDCDLNVVRLCFQVFLPDEHGNLTTALPPVVSNPIYDNRAPNTAELRICRVNKNCGSVRGGDEIFLLCDKVQKDDIEVRFVLNDWEAKGIFSQADVHRQVAIVFKTPPYCKAITEPVTVKMQLRRPSDQEVSESMDFRYLPDEKDTYGNKAKKQKTTLLFQKLCQDHVETGFRHVDQDGLELLTSGDPPTLASQSAGITVNFPERPRPGLLGSIGEGRYFKKEPNLFSHDAVVREMPTGVSSQAESYYPSPGPISSGLSHHASMAPLPSSSWSSVAHPTPRSGNTNPLSSFSTRTLPSNSQGIPPFLRIPVGNDLNASNACIYNNADDIVGMEASSMPSADLYGISDPNMLSNCSVNMMTTSSDSMGETDNPRLLSMNLENPSCNSVLDPRDLRQLHQMSSSSMSAGANSNTTVFVSQSDAFEGSDFSCADNSMINESGPSNSTNPNSHGFVQDSQYSGIGSMQNEQLSDSFPYEFFQV

SEQ ID NO: 4 - protein, 756 aa, Homo sapiens
MSWSPSLTTQTCGAWEMKERLGTGGFGNVIRWHNQETGEQIAIKQCRQELSPRNRERWCLEIQIMRRLTHPNVVAARDVPEGMQNLAPNDLPLLAMEYCQGGDLRKYLNQFENCCGLREGAILTLLSDIASALRYLHENRIIHRDLKPENIVLQQGEQRLIHKIIDLGYAKELDQGSLCTSFVGTLQYLAPELLEQQKYTVTVDYWSFGTLAFECITGFRPFLPNWQPVQWHSKVRQKSEVDIVVSEDLNGTVKFSSSLPYPNNLNSVLAERLEKWLQLMLMWHPRQRGTDPTYGPNGCFKALDDILNLKLVHILNMVTGTIHTYPVTEDESLQSLKARIQQDTGIPEEDQELLQEAGLALIPDKPATQCISDGKLNEGHTLDMDLVFLFDNSKITYETQISPRPQPESVSCILQEPKRNLAFFQLRKVWGQVWHSIQTLKEDCNRLQQGQRAAMMNLLRNNSCLSKMKNSMASMSQQLKAKLDFFKTSIQIDLEKYSEQTEFGITSDKLLLAWREMEQAVELCGRENEVKLLVERMMALQTDIVDLQRSPMGRKQGGTLDDLEEQARELYRRLREKPRDQRTEGDSQEMVRLLLQAIQSFEKKVRVIYTQLSKTVVCKQKALELLPKVEEVVSLMNEDEKTVVRLQEKRQKELWNLLKIACSKVRGPVSGSPDSMNASRLSQPGQLMSQPSTASNSLPEPAKKSEELVAEAHNLCTLLENAIQDTVREQDQSFTALDWSWLQTEEEEHSCLEQAS

SEQ ID NO: 5 - DNA, 50 bp, Homo sapiens
caaccgaacatacccttctatccaggtaatagacccttcttctgtgtcta

SEQ ID NO: 6 - protein, 21 aa, Homo sapiens
MASDYELLWKRKSENYISNKE

SEQ ID NO: 7 - DNA, 25 bp, Artificial
  Synthetic splice switching antisense oligonucleotide for c-Rel
acagaagaagggtctattacctgga

SEQ ID NO: 8 - DNA, 50 bp, Homo sapiens
gggacagacgactgggggcgctcagtttccagaacctgggaatccagtgt

SEQ ID NO: 9 - DNA, 23 bp, Artificial
  Synthetic splice switching antisense oligonucleotide for RELA
gaaactgagcgcccccagtcgtc

SEQ ID NO: 10 - DNA, 25 bp, Artificial
  Synthetic splice switching antisense oligonucleotide for RELB
cggagctgcaggaagagaagtcctc

SEQ ID NO: 11 - DNA, 25 bp, Artificial
  Synthetic splice switching antisense oligonucleotide for IKK2 (IKK2-ex3-3′SS)
gtttcctacagaaacagcacagtgg

SEQ ID NO: 12 - DNA, 25 bp, Artificial
  Synthetic splice switching antisense oligonucleotide for IKK2 (IKK2-ex5-5′SS)
ggttcaggtactgtcaagaaagtga

SEQ ID NO: 13 - DNA, 25 bp, Artificial
  Synthetic irrelevant Control SSO
cctcttacctcagttacaatttata

SEQ ID NO: 14 - DNA, 20 bp, Artificial
  Synthetic primer
ggcctcctgactgactgact

SEQ ID NO: 15 - DNA, 21 bp, Artificial
  Synthetic primer
gtagccgtctctgcagtcttt

SEQ ID NO: 16 - DNA, 20 bp, Artificial
  Synthetic primer
gcgagaggagcacagatacc

SEQ ID NO: 17 - DNA, 20 bp, Artificial
  Synthetic primer
tcactcggcagatcttgagc

SEQ ID NO: 18 - DNA, 18 bp, Artificial
  Synthetic primer
ggcctccggtgcgtataa

SEQ ID NO: 19 - DNA, 20 bp, Artificial
  Synthetic primer
tgttcggttgttgtctgtgc

SEQ ID NO: 20 - DNA, 20 bp, Artificial
  Synthetic primer
agccatggcctccgattatg

SEQ ID NO: 21 - DNA, 20 bp, Artificial
  Synthetic primer
aaggtctgcgttcttgtcca

(19) United States
(12) Patent Application Publication
(10) Pub. No.: US 2023/0365964 A1
(43) Pub. Date: Nov. 16, 2023

(54) USE OF SPLICE SWITCHING OLIGONUCLEOTIDES FOR EXON SKIPPING-MEDIATED KNOCKDOWN OF NF-KB COMPONENTS IN B CELLS

(71) Applicants: INSERM (INSTITUT NATIONAL DE LA SANTÈ ET DE LA RECHERCH MÉDICALE, Paris (FR); UNIVERSITÉ DE LIMOGES, Limoges (FR); CENTRE HOSPITALIER RÉGIONAL UNIVERSITAIRE DE LIMOGES, Limoges (FR); CENTRE NATIONAL DE LA RECHERCHE SCIENTIFIQUE, Paris Cedex 16 (FR)

(72) Inventors: Laurent DELPY, LIMOGES (FR); Anne MARCHALOT, LIMOGES (FR); Jean FEUILLARD, LIMOGES (FR); Francois BOYER, LIMOGES (FR); Nathalie FAUMONT, LIMOGES (FR)

(21) Appl. No.: 18/044,698

(22) PCT Filed: Sep. 9, 2021
(86) PCT No.: PCT/EP2021/074745

§ 371 Date: Mar. 9, 2023

(30) Foreign Application Priority Data
Sep. 10, 2020 (EP) 20306003.3

(51) Int. Cl. C12N 15/113

(52) U.S. Cl. CPC C12N 15/113; C12N 2310/11; C12N 2320/33

(57) ABSTRACT

The need to identify new therapeutic approaches in the treatment of cancers of the B lymphoid lineage is crucial. Here, the inventors provide evidence for efficient knockdown of c-REL and RELA expression after treatment with splice switching antisense oligonucleotides (SSO) inducing exon skipping and reading frameshift. For instance, treatments with morpholino SSO targeting c-REL exon 2 donor splice site or RELA exon 5 acceptor splice site elicited very efficient knockdown in diffuse large B cell lymphoma (DLBCL) cell lines and antibody-secreting cells derived from primary human B cells. Consistent with the clinical relevance of c-REL activation in DLBCL, treatment with c-REL SSO induced major alterations in NF-κB and TNF signalling pathways and strongly decreased cell viability. Altogether, SSO-mediated knockdown is a powerful approach to inhibit transiently the expression of a NF-κB component in B-lineage cells that should open new avenues for cancer treatments. Accordingly, the present invention relates to the use of splice switching oligonucleotides for exon skipping-mediated knockdown of a NF-κB component in B cells.

### FIELD OF THE INVENTION

The present invention is in the field of medicine, in particular oncology.

### BACKGROUND OF THE INVENTION

B cells and plasma cells are key actors in humoral immune response allowing antibody production and long-term defence against pathogens such as virus, bacteria or fungus. B cells are activated when an extracellular antigen binds their B cell receptor (BCR) which induces rapid cell proliferation and differentiation into antibody secreting cells (ASC), either plasmablasts or plasma cells, to allow a sustainable antibody production (1). However, even if this mechanism is well described in literature, some questions remains concerning the B cell differentiation process in humans, notably how plasma cells are homed to their bone marrow niches (2), how high affinity clones are selected (3) and what mechanism lies under their extended longevity (4,5). Unravelling these questions could give new leads to treat antibody-mediated diseases such as allergy, autoimmunity or multiple myeloma and even benefit vaccine development. Studying plasma cell differentiation with human in vitro models rather than mouse models that showed significant differences in this matter (6) and where some ASC related diseases still cannot be fully reproduced (7-9) seems to be the best way to go. However, gene knockdown in primary human plasma cells and B cells has always been a struggle, impeding research progress on non-cancer human B cells and plasma cell studies. Indeed, RNAi mediated gene knockdown in primary B cell is known to induce high mortality rates and poor penetrance during transfection, responsible for a lower knockdown efficiency compared to transformed B cells and plasma cells (10). Recently, it was demonstrated that CRISPR-Cas9 technology is efficient for gene knockout on expended primary B cells and plasma cell differentiation models (11,12) but additional tools to realize a transient knockdown and avoid DNA modifications that could affect other genes (13,14) are still needed.

Antisense oligonucleotides (ASO) are a short single chain of modified DNA that can be used to degrade mRNA by RNAse H1 recruitment or to modify pre-mRNA splicing among others mechanisms, depending of the oligonucleotide chemistry (for review see (15,16)). In addition to RNAse H1-dependant RNA degradation, splice-switching oligonucleotides (SSO) can also be designed to knockdown gene expression, as shown for STAT3 by Zammarchi and colleagues (17). These authors described that splice switching oligonucleotides (SSO) could be used to induce forced splicing-dependent nonsense-mediated decay (FSD-NMD), a mechanism based on SSO-mediated exon skipping to create a reading frameshift, the apparition of a premature stop codon (PTC) and ultimately the degradation of alternative mRNAs by NMD (17). While any splicing modulation producing a reading frameshift and the appearance of PTC can be suitable for FSD-NMD, exon-skipping is the most frequent alternative splicing event in humans (18), and can be easily achieved with SSO hybridizing to either the donor splice site (5′SS), the acceptor splice site (3′SS) or exonic or intronic splicing enhancer (ESE or ISE) sequences (19). However, to efficiently knockdown gene expression, the delivery of sufficient SSO amounts into intracellular compartments remains challenging, as for RNAi technology. Over the last decades, numerous strategies have been developed to overcome this limitation and molecules such as peptide/ligand conjugates, nanoparticles or adeno-associated virus (AAV) have been used to improve the delivery of antisense compounds (20). However, the development of such FSD-NMD strategies to modulate gene expression in B-lineage cells, including primary B or plasma cells, is still lacking.

During plasma cell differentiation, some key genes are differentially regulated like Bcl-6 and Blimp1 expressed in B cells and plasma cells respectively (21). Among those genes, NF-κB components such as c-REL and RELA are respectively down- and up-regulated during plasma cell differentiation, Blimp1 being upregulated by RELA expression which in turn, downregulates c-REL expression by binding its enhancer in ASC (22). Moreover, c-REL and RELA expression is upregulated in many cancers, especially of B cell lineage origin (23,24). Therefore, c-REL and RELA could be interesting targets to cure B cells and plasma cells malignancies like diffuse large B cell lymphoma (DLBCL) or multiple myeloma (MM).

### SUMMARY OF THE INVENTION

The present invention is defined by the claims. In particular, the present invention relates to the use of splice switching oligonucleotides for exon skipping-mediated knockdown of a NF-κB component in B cells.

### DETAILED DESCRIPTION OF THE INVENTION

The need to identify new therapeutic approaches in the treatment of cancers of the B lymphoid lineage is crucial. Unlike CRISPR/Cas technology, antisense strategies allow transient modification of gene expression and lack mutagenic effects at the DNA level. Here, the inventors provide evidence for efficient knockdown of c-REL and RELA expression after treatment with splice switching antisense oligonucleotides (SSO) inducing exon skipping and reading frameshift. They also developed a tool to facilitate the choice of exons for on purpose inhibition of mouse and human gene expression. Interestingly, treatments with morpholino SSO targeting c-REL exon 2 donor splice site or RELA exon 5 acceptor splice site elicited very efficient knockdown in diffuse large B cell lymphoma (DLBCL) cell lines and antibody-secreting cells derived from primary human B cells. Consistent with the clinical relevance of c-REL activation in DLBCL, treatment with c-REL SSO induced major alterations in NF-κB and TNF signalling pathways and strongly decreased cell viability. Altogether, SSO-mediated knockdown is a powerful approach to inhibit transiently the expression of a NF-κB component in B-lineage cells that should open new avenues for cancer treatments.

### Main Definitions

As used herein, the term “subject” refers to any mammals, such as a rodent, a feline, a canine, and a primate.

As used herein, the term “B cell” has its general meaning in the art and is used herein to mean an immune cell that develops in the bone marrow and is highly specialized for making immunoglobulins and antibodies. A B cell is a lymphocyte which is derived from bone marrow and provides humoral immunity. A B cell recognizes antigen molecules in solution and matures into a plasma cell. Thus, when the term “B cell” is used herein it is intended to encompass cells developed from B cells such as plasma cells.

As used herein, the term “plasma cell” has its general meaning in the art and is intended to mean a cell that develops from a B lymphocyte in reaction to a specific antigen. Plasma cells are found in bone marrow and blood. A plasma cell may also be called a plasma B cell or plasmacyte and are cells in the immune system which secrete large amounts of antibodies. Plasma cells differentiate from B cells upon stimulation by CD4+ lymphocytes. A plasma cell is a type of white blood cell that produces antibodies and is derived from an antigen-specific B cell.

As used herein, the terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “NF-κB” has its general meaning in the art and refers to the nuclear factor kappa-light-chain-enhancer of activated B cells. NF-κB is a protein complex that controls transcription of DNA, cytokine production and cell survival.

As used herein, the term “NF-κB component” refers to a protein that has a Rel homology domain in their N-terminus. A subfamily of NF-κB proteins, including RELA, RelB, and c-REL, have a transactivation domain in their C-termini. In contrast, the NF-κB1 and NF-κB2 proteins are synthesized as large precursors, p105, and p100, which undergo processing to generate the mature NF-κB subunits, p50 and p52, respectively. The processing of p105 and p100 is mediated by the ubiquitin/proteasome pathway and involves selective degradation of their C-terminal region containing ankyrin repeats. Whereas the generation of p52 from p100 is a tightly regulated process, p50 is produced from constitutive processing of p105.

As used herein, the term “RELA” has its general meaning in the art and refers to the transcription factor p65. An exemplary amino acid sequence for RELA is shown as SEQ ID NO:1.

>sp|Q04206|TF65_HUMAN Transcription factor p65 OS = Homo sapiens
OX = 9606 GN = RELA PE = 1 SV = 2
SEQ ID NO: 1
MDELFPLIFPAEPAQASGPYVEIIEQPKQRGMRFRYKCEGRSAGSIPGERSTDTTKTHPT
IKINGYTGPGTVRISLVTKDPPHRPHPHELVGKDCRDGFYEAELCPDRCIHSFQNLGIQC
VKKRDLEQAISQRIQTNNNPFQVPIEEQRGDYDLNAVRLCFQVTVRDPSGRPLRLPPVLS
HPIFDNRAPNTAELKICRVNRNSGSCLGGDEIFLLCDKVQKEDIEVYFTGPGWEARGSFS
QADVHRQVAIVFRTPPYADPSLQAPVRVSMQLRRPSDRELSEPMEFQYLPDTDDRHRIEE
KRKRTYETFKSIMKKSPFSGPTDPRPPPRRIAVPSRSSASVPKPAPQPYPFTSSLSTINY
DEFPTMVFPSGQISQASALAPAPPQVLPQAPAPAPAPAMVSALAQAPAPVPVLAPGPPQA
VAPPAPKPTQAGEGTLSEALLQLQFDDEDLGALLGNSTDPAVFTDLASVDNSEFQQLLNQ
GIPVAPHTTEPMLMEYPEAITRLVTGAQRPPDPAPAPLGAPGLPNGLLSGDEDFSSIADM
DFSALLSQISS

As used herein, the term “RELB” has its general meaning in the art and refers to the Transcription factor RelB. An exemplary amino acid sequence for RELB is shown as SEQ ID NO:2.

>sp|Q01201|RELB_HUMAN Transcription factor RelB OS = Homo sapiens
OX = 9606 GN = RELB PE = 1 SV = 2
SEQ ID NO: 2
MLRSGPASGPSVPTGRAMPSRRVARPPAAPELGALGSPDLSSLSLAVSRSTDELEIIDEY
IKENGFGLDGGQPGPGEGLPRLVSRGAASLSTVTLGPVAPPATPPPWGCPLGRLVSPAPG
PGPQPHLVITEQPKQRGMRFRYECEGRSAGSILGESSTEASKTLPAIELRDCGGLREVEV
TACLVWKDWPHRVHPHSLVGKDCTDGICRVRLRPHVSPRHSFNNLGIQCVRKKEIEAAIE
RKIQLGIDPYNAGSLKNHQEVDMNVVRICFQASYRDQQGQMRRMDPVLSEPVYDKKSTNT
SELRICRINKESGPCTGGEELYLLCDKVQKEDISVVFSRASWEGRADFSQADVHRQIAIV
FKTPPYEDLEIVEPVTVNVFLQRLTDGVCSEPLPFTYLPRDHDSYGVDKKRKRGMPDVLG
ELNSSDPHGIESKRRKKKPAILDHFLPNHGSGPFLPPSALLPDPDFFSGTVSLPGLEPPG
GPDLLDDGFAYDPTAPTLFTMLDLLPPAPPHASAVVCSGGAGAVVGETPGPEPLTLDSYQ
APGPGDGGTASLVGSNMFPNHYREAAFGGGLLSPGPEAT

As used herein, the term “c-REL” has its general meaning in the art and refers to the Proto-oncogene c-REL. An exemplary amino acid sequence for c-REL is shown as SEQ ID NO:3.

>sp|Q04864|REL_HUMAN Proto-oncogene c-REL OS = Homo sapiens
OX = 9606 GN = REL PE = 1 SV = 1
SEQ ID NO: 3
MASGAYNPYIEIIEQPRQRGMRFRYKCEGRSAGSIPGEHSTDNNRTYPSIQIMNYYGKGK
VRITLVTKNDPYKPHPHDLVGKDCRDGYYEAEFGQERRPLFFQNLGIRCVKKKEVKEAII
TRIKAGINPFNVPEKQLNDIEDCDLNVVRLCFQVFLPDEHGNLTTALPPVVSNPIYDNRA
PNTAELRICRVNKNCGSVRGGDEIFLLCDKVQKDDIEVRFVLNDWEAKGIFSQADVHRQV
AIVFKTPPYCKAITEPVTVKMQLRRPSDQEVSESMDFRYLPDEKDTYGNKAKKQKTTLLF
QKLCQDHVETGFRHVDQDGLELLTSGDPPTLASQSAGITVNFPERPRPGLLGSIGEGRYF
KKEPNLFSHDAVVREMPTGVSSQAESYYPSPGPISSGLSHHASMAPLPSSSWSSVAHPTP
RSGNTNPLSSFSTRTLPSNSQGIPPFLRIPVGNDLNASNACIYNNADDIVGMEASSMPSA
DLYGISDPNMLSNCSVNMMTTSSDSMGETDNPRLLSMNLENPSCNSVLDPRDLRQLHQMS
SSSMSAGANSNTTVFVSQSDAFEGSDFSCADNSMINESGPSNSTNPNSHGFVQDSQYSGI
GSMQNEQLSDSFPYEFFQV

As used herein, the term “IKK2” has its general meaning in the art and refers to the Inhibitor of nuclear factor kappa-B kinase subunit beta. An exemplary amino sequence for IKK2 is shown as SEQ ID NO:4.

>sp|O14920|IKKB_HUMAN Inhibitor of nuclear factor kappa-B kinase
subunit beta OS = Homo sapiens OX = 9606 GN = IKBKB PE = 1 SV = 1
SEQ ID NO: 4
MSWSPSLTTQTCGAWEMKERLGTGGFGNVIRWHNQETGEQIAIKQCRQELSPRNRERWCL
EIQIMRRLTHPNVVAARDVPEGMQNLAPNDLPLLAMEYCQGGDLRKYLNQFENCCGLREG
AILTLLSDIASALRYLHENRIIHRDLKPENIVLQQGEQRLIHKIIDLGYAKELDQGSLCT
SFVGTLQYLAPELLEQQKYTVTVDYWSFGTLAFECITGFRPFLPNWQPVQWHSKVRQKSE
VDIVVSEDLNGTVKFSSSLPYPNNLNSVLAERLEKWLQLMLMWHPRQRGTDPTYGPNGCF
KALDDILNLKLVHILNMVTGTIHTYPVTEDESLQSLKARIQQDTGIPEEDQELLQEAGLA
LIPDKPATQCISDGKLNEGHTLDMDLVFLFDNSKITYETQISPRPQPESVSCILQEPKRN
LAFFQLRKVWGQVWHSIQTLKEDCNRLQQGQRAAMMNLLRNNSCLSKMKNSMASMSQQLK
AKLDFFKTSIQIDLEKYSEQTEFGITSDKLLLAWREMEQAVELCGRENEVKLLVERMMAL
QTDIVDLQRSPMGRKQGGTLDDLEEQARELYRRLREKPRDQRTEGDSQEMVRLLLQAIQS
FEKKVRVIYTQLSKTVVCKQKALELLPKVEEVVSLMNEDEKTVVRLQEKRQKELWNLLKI
ACSKVRGPVSGSPDSMNASRLSQPGQLMSQPSTASNSLPEPAKKSEELVAEAHNLCTLLE
NAIQDTVREQDQSFTALDWSWLQTEEEEHSCLEQAS

As used herein, the expression “reducing the expression of NF-κB component” means a measurable decrease in the number of said NF-κB component in the B cells of the subject. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of said NF-κB component to an amount below detectable limits. Methods for quantifying expression of NF-κB component are well known in the art and typically include those described in the EXAMPLE.

As used herein, the term “pre-mRNA”, “precursor mRNA” or “primary RNA transcript” refers to a strand of messenger ribonucleic acid (mRNA), synthesized from a DNA template in the nucleus of a cell by transcription, prior to processing events such as splicing. Generally, eukaryotic pre-mRNA exists only briefly before it is fully processed into mature mRNA. Pre-mRNA includes two different types of segments, exons and introns. Most of exons encode protein, while introns are usually excised before translation by a process known as “splicing”.

As used herein, the term “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA.

As used herein, the term “intron” refers to a nucleic acid region (within a gene) that is not translated into a protein. An intron is a non-coding section that is transcribed into a precursor mRNA (pre-mRNA), and subsequently removed by splicing during formation of the mature RNA.

As used herein, the term “splice site” in the context of a pre-mRNA molecule, refers to the short conserved sequence at the 5′ end (donor site) or 3′ end (acceptor site) of an intron to which a spliceosome binds and catalyzes the splicing of the intron from the pre-mRNA.

As used herein, the term “exon skipping” refers generally to the process by which an entire exon, or a portion thereof, is removed from a given pre-processed RNA, and is thereby excluded from being present in the mature RNA. According to the present invention the exon deletion leads to a reading frame shift in the shortened transcribed mRNA that would lead to the generation of truncated non-functional protein or nonsense-mediated decay (NMD) degradation.

As used herein, the term “antisense oligonucleotide” or “ASO” refers to a single strand of DNA, RNA, or modified nucleic acids that is complementary to a chosen sequence. Antisense RNA can be used to prevent protein translation of certain mRNA strands by binding to them. Antisense DNA can be used to target a specific, complementary (coding or non-coding) RNA. Such an antisense oligomer can be designed to block or inhibit translation of mRNA or to inhibit natural pre-mRNA splice processing, and may be said to be “directed to” or “targeted against” a target sequence with which it hybridizes. According to the present invention, the target sequence is a splice site of a pre-processed mRNA. In said embodiments, the ASO is named as a “splice switching antisense oligonucleotide” or “SSO”. For instance, the target sequence for a splice site may include an mRNA sequence having its 5′ end 1 to about 25 base pairs downstream of a normal splice acceptor junction in a preprocessed mRNA. A preferred target sequence is any region of a precursor mRNA that includes a splice site or is contained entirely within an exon coding sequence or spans a splice acceptor or donor site or exon/intron regulatory sequences (ESE, ISE).

As used herein, the term “complementary” as used herein includes “fully complementary” and “substantially complementary”, meaning there will usually be a degree of complementarity between the oligonucleotide and its corresponding target sequence of more than 80%, preferably more than 85%, still more preferably more than 90%, most preferably more than 95%. For example, for an oligonucleotide of 20 nucleotides in length with one mismatch between its sequence and its target sequence, the degree of complementarity is 95%.

As used herein, the term “isolated” means material that is substantially or essentially free from components that normally accompany it in its native state. For example, an “isolated polynucleotide,” as used herein, may refer to a polynucleotide that has been purified or removed from the sequences that flank it in a naturally-occurring state, e.g., a DNA fragment that has been removed from the sequences that are normally adjacent to the fragment.

As used herein, the term “stabilized SSO” refers to a SSO that is relatively resistant to in vivo degradation (e.g. via an exo- or endo-nuclease).

As used herein, the term “B-cell malignancy” includes any type of leukemia or lymphoma of B cells.

As used herein, the term “B cell lymphoma” refers to a cancer that arises in cells of the lymphatic system from B cells.

As used herein, the term “multiple myeloma” as used herein means a disseminated malignant neoplasm of plasma cells which is characterized by multiple bone marrow tumor foci and secretion of an M component (a monoclonal immunoglobulin fragment), associated with widespread osteolytic lesions resulting in bone pain, pathologic fractures, hypercalcaemia and normochromic normocytic anaemia.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” is intended for a minimal amount of the active agent (i.e the SSO of the present invention) which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder.

### Methods of the Present Invention

An object of the present invention relates to a method of reducing the expression of a NF-κB component in the B cells of a subject in need thereof comprising administering to the subject an effective amount of at least one splice switching antisense oligonucleotide targeting a splice site of one exon, or a splicing regulatory sequence in the pre-mRNA molecule encoding for the NF-κB component to alter splicing by blocking the recognition of said splice site by splicing machinery and thus inducing the exon skipping.

According to the present invention the splice switching oligonucleotide (SSO) mediates the exon-skipping for a pre-mRNA having at least 3 exons with a targeted internal one having a number of nucleotides not divisible by 3 for inducing a reading frameshift. In addition, the exon skipping must provoke the appearance of a premature termination codon (PTC) to shorten drastically the open reading frame and/or support NMD degradation. Typically, the splice switching oligonucleotide (SSO) of the present invention is designed according to the method disclosed in the EXAMPLE and as depicted in FIG. 1.

In some embodiments, the splice switching antisense oligonucleotide of the present invention is an antisense RNA.

In some embodiments, the splice switching antisense oligonucleotide of the present invention is an antisense DNA.

The length of the splice switching antisense oligonucleotide may vary so long as it is capable of binding selectively to the intended location within the pre-mRNA molecule. The length of such sequences can be determined in accordance with selection procedures described herein. Generally, the antisense molecule will be from about 10 nucleotides in length up to about 50 nucleotides in length. It will be appreciated however that any length of nucleotides within this range may be used in the method. Preferably, the length of the antisense molecule is between 10-30 nucleotides in length. In some embodiments, the splice switching antisense oligonucleotide of the present invention has a sufficient length. As used herein, “sufficient length” refers to an antisense oligonucleotide that is complementary to at least 8, more typically 8-30, contiguous nucleobases in the target pre-mRNA. In some embodiments, an antisense of sufficient length includes at least 8, 9, 10, 11, 12, 13, 14, 15, 17, 20 or more contiguous nucleobases in the target pre-mRNA. In some embodiments an antisense of sufficient length includes at least 16, 17, 18, 19, 20, 21, 22, 23, 24, or 25 contiguous nucleobases in the target pre-mRNA.

In some embodiments, the method of the present invention is particularly suitable for reducing the expression of c-REL in the B cells of the subject. In some embodiments, the splice switching antisense oligonucleotide of the present invention targets the c-REL exon 2 donor splice site. In some embodiments, the splice switching antisense oligonucleotide of the present invention is complementary to the nucleic acid sequence as shown in SEQ ID NO:5. In some embodiments, the splice switching antisense oligonucleotide of the present invention converts the c-Rel protein translation into an inactive peptide of 21 amino acids, lacking all active domains compared to full-length c-Rel isoform. In some embodiments, the inactive peptide consists of the amino acid sequence as set forth in SEQ ID NO:6. In some embodiments, the splice switching antisense oligonucleotide of the present invention targets the c-REL exon 2 donor splice site and comprises the sequences as set forth in SEQ ID NO:7.

target sequence for c-Rel (25 nt exon + 25 nt intron)
SEQ ID NO: 5
5′-caaccgaacatacccttctatccaggtaatagacccttcttctgtgtcta-3′
inactive peptide of 21 amino acids
SEQ ID NO: 6
MASDYELLWKRKSENYISNKE* (* = stop codon)
splice switching antisense oligonucleotide for c-Rel
SEQ ID NO: 7
5′-ACAGAAGAAGGGTCTATTACCTGGA-3

In some embodiments, the method of the present invention is particularly suitable for reducing the expression of RELA in the B cells of the subject. In some embodiments, the splice switching antisense oligonucleotide of the present invention targets the RELA acceptor splice site of exon 5. In some embodiments, the splice switching antisense oligonucleotide of the present invention is complementary to the nucleic acid sequence as shown in SEQ ID NO:8. In some embodiments, the splice switching antisense oligonucleotide of the present invention targets the RELA acceptor splice site of exon 5 and comprises the sequence as set forth in SEQ ID NO:9.

SEQ ID NO: 8 target sequence for RelA (25 nt intron + 25 nt EXON):
5′-gggacagacgactgggggcgctcagTTTCCAGAACCTGGGAATCCAGTGT-3′
splice switching antisense oligonucleotide for RELA
SEQ ID NO: 9
5′-GAAACTGAGCGCCCCCAGTCGTC-3′

In some embodiments, the method of the present invention is particularly suitable for reducing the expression of RELB in the B cells of the subject. In some embodiments, the splice switching antisense oligonucleotide of the present invention targets the RELB acceptor splice site of exon 5. In some embodiments, the splice switching antisense oligonucleotide of the present invention targets the RELB acceptor splice site of exon 5 and comprises the sequence as set forth in SEQ ID NO:10.

splice switching antisense oligonucleotide for RELB
SEQ ID NO: 10
5′-CGGAGCTGCAGGAAGAGAAGTCCTC-3′

In some embodiments, the method of the present invention is particularly suitable for reducing the expression of IKK2 in the B cells of the subject. In some embodiments, the splice switching antisense oligonucleotide of the present invention targets the IKK2 acceptor splice site of exon 3 or exon 5. In some embodiments, the splice switching antisense oligonucleotide of the present invention targets the IKK2 acceptor splice site of exon 3 and comprises the sequence as set forth in SEQ ID NO:11. In some embodiments, the splice switching antisense oligonucleotide of the present invention targets the IKK2 acceptor splice site of exon 5 and comprises the sequence as set forth in SEQ ID NO:12.

SEQ ID NO: 11 splice switching antisense oligonucleotide for IKK2
(IKK2-ex3-3′SS):
5′-GTTTCCTACAGAAACAGCACAGTGG-3′
SEQ ID NO: 12 splice switching antisense oligonucleotide for IKK2
(IKK2-ex5-3′SS):
5′-GGTTCAGGTACTGTCAAGAAAGTGA-3′

In some embodiments, the splice switching antisense oligonucleotide of the present invention is stabilized. Stabilization can be a function of length or secondary structure. Alternatively, SSO stabilization can be accomplished via phosphate backbone modifications. Preferred stabilized SSOs of the present invention have a modified backbone, e.g. have phosphorothioate linkages to provide maximal activity and protect the SSO from degradation by intracellular exo- and endo-nucleases. Other possible stabilizing modifications include phosphodiester modifications, combinations of phosphodiester and phosphorothioate modifications, methylphosphonate, methylphosphorothioate, phosphorodithioate, p-ethoxy, and combinations thereof. Chemically stabilized, modified versions of the SSO's also include “Morpholinos” (phosphorodiamidate morpholino oligomers, PMOs), 2′-O-Met oligomers, 2′Methoxy-ethyl oligomers, 2′-Fluoro (2′-F) oligomers, tricyclo (tc)-DNAs, U7 short nuclear (sn) RNAs, tricyclo-DNA-oligoantisense molecules (U.S. Provisional Patent Application Ser. No. 61/212,384 For: Tricyclo-DNA Antisense Oligonucleotides, Compositions and Methods for the Treatment of Disease, filed Apr. 10, 2009, the complete contents of which is hereby incorporated by reference, unlocked nucleic acid (UNA), locked nucleic acid (LNA), peptide nucleic acid (PNA), serinol nucleic acid (SNA), twisted intercalating nucleic acid (TINA), anhydrohexitol nucleic acid (HNA), cyclohexenyl nucleic acid (CeNA), D-altritol nucleic acid (ANA) and morpholino nucleic acid (MNA) have also been investigated in splice modulation. Recently, nucleobase-modified AOs containing 2-thioribothymidine, and 5-(phenyltriazol)-2-deoxyuridine nucleotides have been reported to induce exon skipping (Chen S, Le B T, Chakravarthy M, Kosbar T R, Veedu R N. Systematic evaluation of 2′-Fluoro modified chimeric antisense oligonucleotide-mediated exon skipping in vitro. Sci Rep. 2019 Apr. 15; 9(1):6078.). In some embodiments, the antisense oligonucleotides of the invention may be 2′-O-Me RNA/ENA chimera oligonucleotides (Takagi M, Yagi M, Ishibashi K, Takeshima Y, Surono A, Matsuo M, Koizumi M. Design of 2′-O-Me RNA/ENA chimera oligonucleotides to induce exon skipping in dystrophin pre-mRNA. Nucleic Acids Symp Ser (Oxf). 2004; (48):297-8). Other forms of SSOs that may be used to this effect are SSO sequences coupled to small nuclear RNA molecules such as U1 or U7 in combination with a viral transfer method based on, but not limited to, lentivirus or adeno-associated virus (Denti, M A, et al, 2008; Goyenvalle, A, et al, 2004). In some embodiments, the antisense oligonucleotides of the invention are 2′-O-methyl-phosphorothioate nucleotides.

The SSOs of the invention can be synthesized de novo using any of a number of procedures well known in the art. For example, the b-cyanoethyl phosphoramidite method (Beaucage et al., 1981); nucleoside H-phosphonate method (Garegg et al., 1986; Froehler et al., 1986, Garegg et al., 1986, Gaffney et al., 1988). These chemistries can be performed by a variety of automated nucleic acid synthesizers available in the market. These nucleic acids may be referred to as synthetic nucleic acids. Alternatively, SSO's can be produced on a large scale in plasmids (see Sambrook, et al., 1989). SSO's can be prepared from existing nucleic acid sequences using known techniques, such as those employing restriction enzymes, exonucleases or endonucleases. SSO's prepared in this manner may be referred to as isolated nucleic acids.

In some embodiments, the splice switching antisense oligonucleotide of the present invention may be delivered in vivo alone or in association with a vector. In its broadest sense, a “vector” is any vehicle capable of facilitating the transfer of the splice switching antisense oligonucleotide of the present invention to the cells. Preferably, the vector transports the nucleic acid to cells with reduced degradation relative to the extent of degradation that would result in the absence of the vector. In general, the vectors useful in the invention include, but are not limited to, naked plasmids, non-viral delivery systems (electroporation, sonoporation, cationic transfection agents, liposomes, nanoparticules, peptide-bound SSO, etc . . . ), phagemids, viruses, other vehicles derived from viral or bacterial sources that have been manipulated by the insertion or incorporation of the antisense oligonucleotide nucleic acid sequences. Viral vectors are a preferred type of vector and include, but are not limited to nucleic acid sequences from the following viruses: RNA viruses such as a retrovirus (as for example moloney murine leukemia virus and lentiviral derived vectors), harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus. One can readily employ other vectors not named but known to the art. Typically, viral vectors according to the invention include adenoviruses and adeno-associated (AAV) viruses, which are DNA viruses that have already been approved for human use in gene therapy. Actually 12 different AAV serotypes (AAV1 to 12) are known, each with different tissue tropisms (Wu, Z Mol Ther 2006; 14:316-27). Recombinant AAV are derived from the dependent parvovirus AAV (Choi, V W J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hematopoietic cells; and lack of superinfection inhibition thus allowing multiple series of transductions. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion. Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by, intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation. In some embodiments, the antisense oligonucleotide nucleic acid sequence is under the control of a heterologous regulatory region, e.g., a heterologous promoter. The promoter can also be, e.g., a viral promoter, such as CMV promoter or any synthetic promoters.

The method of the present invention is particularly suitable for the treatment of B cell malignancies. B-cell malignancies include, but are not limited to, non-Hodgkin's lymphoma, Burkitt's lymphoma, small lymphocytic lymphoma, primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL) and related lymphomas (e.g. Waldenstrom's macroglobulinaemia (also called lymphoplasmacytic lymphoma or immunocytoma) or central nervous system lymphomas), leukemias (e.g. acute lymphoblastic leukemia (ALL), chronic lymphocytic leukemia (CLL; also termed B cell chronic lymphocytic leukemia BCLL), hairy cell leukemia and chronic myoblastic leukemia) and myelomas (e.g. multiple myeloma). Additional B cell malignancies include, lymphoplasmacytic lymphoma, plasma cell myeloma, solitary plasmacytoma of bone, extraosseous plasmacytoma, extra-nodal marginal zone B cell lymphoma of mucosa-associated (MALT) lymphoid tissue, nodal marginal zone B cell lymphoma, follicular lymphoma, mantle cell lymphoma, mediastinal (thymic) large B cell lymphoma, intravascular large B cell lymphoma, primary effusion lymphoma, Burkitt's lymphoma/leukemia, grey zone lymphoma, B cell proliferations of uncertain malignant potential, lymphomatoid granulomatosis, and post-transplant lymphoproliferative disorder.

More particularly, the method of the present invention is suitable for the treatment of multiple myeloma.

The method of the present invention is also particularly suitable for the treatment of diseases associated to autoimmunity or inflammation. Examples of said diseases include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, diabetic large-artery disorder, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia pemiciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant-cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant-cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritis, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis.

It will be understood that the total daily usage of the compounds of the present invention (i.e. the SSO of the present invention) will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the splice switching antisense oligonucleotide of the present invention is administered in the form of a pharmaceutical composition. Pharmaceutical compositions of the present invention may also include a pharmaceutically or physiologically acceptable carrier such as saline, sodium phosphate, etc. The compositions will generally be in the form of a liquid, although this need not always be the case. Suitable carriers, excipients and diluents include lactose, dextrose, sucrose, sorbitol, mannitol, starches, gum acacia, calcium phosphates, alginate, tragacanth, gelatin, calcium silicate, microcrystalline cellulose, polyvinylpyrrolidone, celluose, water syrup, methyl cellulose, methyl and propylhydroxybenzoates, mineral oil, etc. The formulations can also include lubricating agents, wetting agents, emulsifying agents, preservatives, buffering agents, etc. Those of skill in the art will also recognize that nucleic acids are often delivered in conjunction with lipids (e.g. cationic lipids or neutral lipids, or mixtures of these), frequently in the form of liposomes or other suitable micro- or nano-structured material (e.g. micelles, lipocomplexes, dendrimers, emulsions, cubic phases, nanoparticules, etc.).

A further object of the present invention relates to a splice switching antisense oligonucleotide as described above. In some embodiments, the splice switching antisense oligonucleotide comprises the sequence as set forth in SEQ ID NO:7, SEQ ID NO:9, SEQ ID NO:10, SEQ ID NO:11 or SEQ ID NO:12.

The invention will be further illustrated by the following figures and examples. However, these examples and figures should not be interpreted in any way as limiting the scope of the present invention.

### FIGURES

FIG. 1. “Exon skipper pipeline” facilitates selection of splice switching oligonucleotides (SSO) to knockdown gene expression. Schematic representation of in-silico sequential steps performed to select exon targets and define the best SSO position for efficient inhibition of gene expression.

FIG. 2. c-Rel exon 2 donor splice site targeting vivo-morpholino SSO allows efficient c-Rel knockdown in DLBCL cell lines and human plasmablasts differentiated from primary B cells. A, Illustration of SSO mediated knockdown of c-Rel transcripts. B-D, SUDHL4 cells were treated 48 hours with 0.25 to 0.75 μM of c-Rel exon 2 donor splice site targeting SSO (SSO) or irrelevant control ASO (CTRL). B, Exon 2 skipping was assessed by RT-PCR C, Knockdown of full length c-Rel mRNAs (left) and the levels of alternative c-Rel mRNAs lacking exon 2 were measured by qRT-PCR D, Knockdown of c-Rel protein was verified by western blot. E-F, SUDHL4 cells were treated 48 hours with 0.5 μM of c-Rel exon 2 donor splice site targeting SSO (SSO) or irrelevant control ASO (CTRL) (n=4). E, Exon 2 skipping was assessed by RT-PCR F, qRT-PCR were performed as in C. G-H, Human plasmablasts differentiated from primary B cells were treated for 72 hours at day 1 following stimulation with 2 μM of SSO or CTRL. G, Exon 2 skipping was assessed by RT-PCR H, Knockdown of c-Rel protein was verified by western blot. ****p<0.0001

FIG. 3. Treatment with c-Rel exon 2 donor splice site targeting SSO reduces viability of DLBCL cells. Apoptosis was analysed by flow cytometry with annexin V and 7-AAD staining. ****p<0.0001

FIG. 4. RelA exon 5 acceptor splice site targeting vivo-morpholino SSO allows protein knockdown of RelA in DLBCL cells and human plasma cells differentiated from primary B cells. A, Illustration of SSO mediated knockdown of RelA transcripts. B-C OCILY10 cells were treated 48 hours with 1.5 to 2 μM of RelA exon 5 acceptor splice site targeting SSO (SSO) or irrelevant control ASO (CTRL). B, Diminution of RelA transcripts was assessed by RT-PCR. C, Knockdown of RelA protein was verified by western blot. D-E Human plasma cells differentiated from primary B cells were treated for 72 hours at day 4 following differentiation with 2 μM of RelA exon 5 acceptor splice site targeting SSO (SSO) or irrelevant control ASO (CTRL). D, Diminution of RelA transcripts was assessed by RT-PCR. E, Knockdown of RelA protein was verified by western blot.

### EXAMPLE

### Methods

### SSO Design

REL and RELA gene sequences were collected from NCBI, GRCh38.p12 assembly, 25 base pairs upstream to 25 base pairs downstream from each splice site. SSO targeting c-REL exon 2 donor splice site (5′-ACAGAAGAAGGGTCTATTACCTGGA-3′) (SEQ ID NO:7), RELA SSO targeting the acceptor splice site of exon 5 (5′-GAAACTGAGCGCCCCCAGTCGTC-3′) (SEQ ID NO:9), and irrelevant Control SSO (5′-CCTCTTACCTCAGTTACAATTTATA-3′) (SEQ ID NO:13) were designed as morpholino or Vivo-morpholino and purchased at Gene Tools, LLC. SSO stock solutions were made at 0.5 mM with sterile nuclease-free water.

### Cell Culture and SSO Treatments

SUDHL4 and OCILY10 (DLBCL) cell lines were cultivated in RPMI1640 medium with Ultraglutamine (Lonza) and 10% or 20% Fetal Bovine Serum (FBS) (Dominique Dutscher) at 37° C. with 5% CO₂ and treated for 48 hours with 0.25 to 0.75 μM SSO for SUDHL4 and with 1.5 to 2 μM SSO for OCILY10. Human Peripheral Blood Mononuclear Cells (PBMC) were obtained by ficoll (Lympholyte-H Celardane) of cytapheresis rings of healthy donors (EFS Bordeaux) and B cells were sorted with the B cell isolation kit II (Miltenyi Biotec). Sorted B cells were cultured for 4 days in RPMI 1640 with UltraGlutamine (Lonza) containing 10% FBS (Dominique Dutscher), 1 mM sodium pyruvate (Eurobio), 1% AANE (Eurobio) and 50 U/ml penicillin/50 μg/ml streptomycin (Gibco) with 1 μg/mL CPG-ODN2006 (Miltenyi), 2.4 μg/mL BCR Fab′2 IgA+G+M (Jackson Immunoresearch), 5 ng/mL hIL-2 (R&D Systems) and 100 ng/mL Mega-CD40 ligand (Enzo Life Sciences) then washed and stimulated in the same media with 5 ng/mL hIL-2 (R&D Systems), 12 ng/mL hIL-10 (R&D Systems) and 5 ng/mL human IL-4 (Peprotech) for 3 more days. Human B cells were treated with 2 μM SSO for 72 hours at day 1 following stimulation for plasmablast study and at day 4 following stimulation for plasma cell study.

### Flow Cytometry

Apoptosis assay was performed on SUDHL4 cells with AnnexinV antibody (BioLegend) and 7-AAD (BD Pharmingen) staining. Data were acquired on a Beckton Dickinson LSRII Fortessa cytometer and analyzed with FlowLogic software.

### Western Blot

Cells were lysed in radioimmunoprecipitation assay (RIPA) buffer (Thermo Scientific) supplemented with protease and phosphatase inhibitor cocktail. Lysates were sonicated and protein concentrations were determined using Pierce™ BCA Protein Assay kit (Thermo Scientific). Proteins were denaturated at 95° C. 5 minutes before separation on SDS-PAGE TGX 12% Stain-Free FastCast Acrylamide (Bio-Rad Laboratories). Proteins were then electro-transferred onto Trans Blot Turbo polyvinylidene fluoride membranes (Bio-Rad Laboratories). Western blots were probed with rabbit anti-human c-REL antibody (Cell Signaling), rabbit anti-human RELA antibody (Cell Signaling) or mouse anti-beta-actin antibody (Sigma). Detection was performed using an HRP-linked goat anti-rabbit or goat anti-mouse secondary antibody (Southern Biotech) and chemiluminescence detection kit (ECL Plus™, GE Healthcare) using ChemiDoc™ Touch Imaging System (Bio-Rad Laboratories).

RT-PCR and qPCRs

Total RNA was prepared using Tri-reagent (Invitrogen) procedures. RT-PCR was carried out on 1 μg DNase I (Invitrogen)-treated RNA using High-Capacity cDNA Reverse Transcription Kit (Applied Biosystem). PCRs were performed on cDNA samples equivalent to 10 to 20 ng of RNA per reaction using the Taq Core Kit (MP Biomedicals) and the primer pair forward 5′-GGCCTCCTGACTGACTGACT (SEQ ID NO:14) and reverse 5′-GTAGCCGTCTCTGCAGTCTTT (SEQ ID NO:15) for c-REL and the primer pair forward 5′-GCGAGAGGAGCACAGATACC (SEQ ID NO:16) and reverse 5′-TCACTCGGCAGATCTTGAGC (SEQ ID NO:17) for RELA. PCRs products were migrated on 2% agarose TBE gels. Quantitative PCRs were performed on cDNA samples equivalent to 5-10 ng of RNA per reaction, using SYBR® Premix Ex Taq™ (Tli RNaseH Plus), ROX plus or Premix Ex Taq™ (Probe qPCR), ROX plus (Takara) on a StepOnePlus Real-Time PCR system (Applied Biosystems). Transcripts were quantified according to the standard 2^{-ΔΔCt} method after normalization to GAPDH (Hs02758991_g1 ThermoFisher Scientific Probe). SYBR quantitative PCR were performed for the quantification of c-REL full length transcripts with forward 5′-GGCCTCCGGTGCGTATAA (SEQ ID NO:18) and reverse 5′-TGTTCGGTTGTTGTCTGTGC (SEQ ID NO:19) primers and for exon 2 skipped c-REL transcripts with forward 5′-AGCCATGGCCTCCGATTATG (SEQ ID NO:20) and reverse 5′-AAGGTCTGCGTTCTTGTCCA (SEQ ID NO:21).

### RNA Sequencing Analysis

Messenger RNA-sequencing was performed on the Illumina NextSeq500 and analyzed with DESeq2 statistical analysis at Nice-Sophia-Antipolis Functional Genomics Platform.

Differentially expressed genes were sorted when the adjusted p value was >0.05 and the fold change was <1.5 or >1.5. Reads were aligned with STAR on the hg38 genome version during the primary analysis REL reads visualization was performed on IGV software (Broad Institute and the Regents of the University of California) and sashimi plot were obtained by ggsashimi tool on R studio software with a cutoff of 5 junctions. Heatmap was obtained with R studio software using ggplot tool on statistically and differentially expressed gene list. Pathways enrichment were analyzed on statistically and differentially expressed gene list with g:Profiler website (25).

### Statistical Analysis

The results are expressed as the mean±standard error of the mean (SEM), and overall differences between variables were evaluated by an unpaired two-tailed Student's t test using Prism GraphPad software (San Diego, CA).

### Results

### Splice Switching Oligonucleotide Selection Workflow for Exon Skipping-Mediated Knockdown of Gene Expression.

Splice switching oligonucleotide (SSO) mediated gene knockdown can be realized with an exon-skipping strategy for the vast majority of human genes, containing ≥3 exons with a targeted internal one not divisible by 3 for a reading frameshift to occur. In addition, exon skipping must provoke the appearance of a PTC to shorten drastically the open reading frame and/or support NMD degradation.

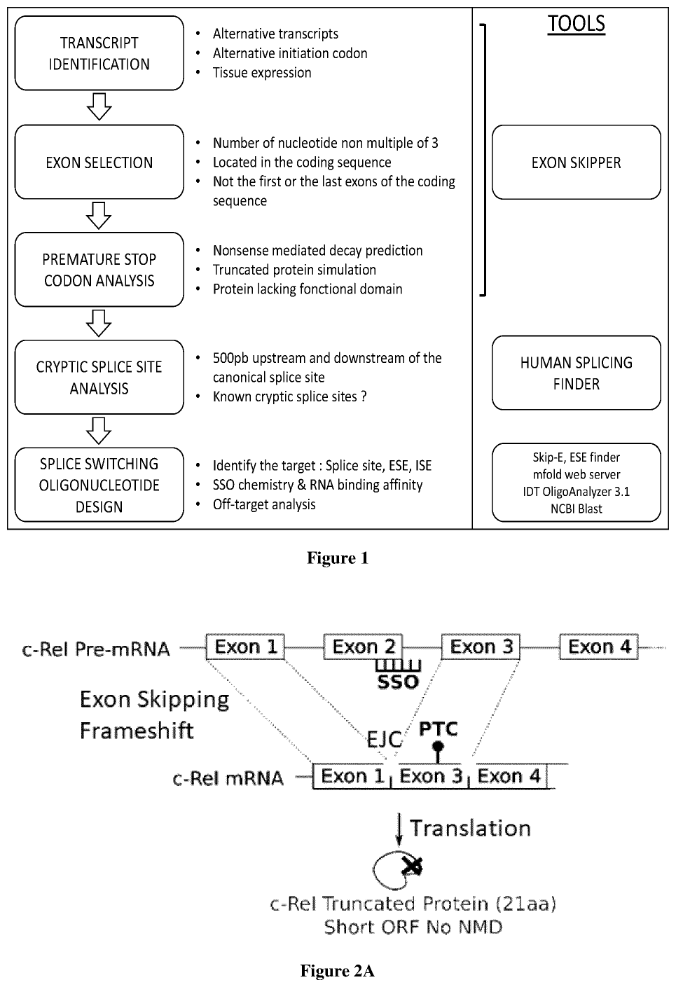

We developed an R shiny tool called Exon Skipper (https://cribl.shinyapps.io/ExonSkipper/) for rapid identification of putative exon candidates for any mouse or human transcripts. This tool also indicates the PTC position for each exon skipping events, providing predictive information with regard to NMD degradation and ORF shortening. For selected exon candidates, we then inspected its nucleotide sequence, together with its surrounding 500 nucleotides intronic sequences, to scrutinize unwanted restoration of the reading frame using cryptic splice sites. This analysis can be made by any splice site scoring tools available like human splicing finder (HSF) (https://hsf.genomnis.com/sequence) (26). The selection of 3′SS targeting SSO is preferred when high risk of cryptic 5′SS that restore reading frame are found and conversely. In the absence of predictive correction of reading frame after alternative splicing involving cryptic splice sites, SSO can be designed to hybridize either 5′SS, 3′SS or ESE/ISE sequences, the latter can be identified using Skip-E (https://skip-e.geneticsandbioinformatics.eu), ESE finder (http://krainer01.cshl.edu/cgi-bin/tools/ESE3/esefinder.cgi) (27) or other tools. Each SSO sequence must be carefully chosen as there is optimal parameters for each oligonucleotide chemistry related to RNA binding affinity (for design tips see (19)). RNA binding parameters can be analysed through IDT OligoAnalyzer (https://eu.idtdna.com/calc/analyzer) and RNA availability through folding RNA simulators such as mfold (http://unafold.rna.albany.edu/?q=mfold) (28). Finally, NCBI BLAST (https://blast.ncbi.nlm.nih.gov/Blast.cgi) analysis of selected SSO sequences towards other transcripts from the same organism is required to exclude SSO with high risk of cross-hybridization to off-targets (FIG. 1). This complete method referred as “Exon skipper pipeline” has been used for the design of SSO targeting two members of the NF-kB pathway, c-Rel and RelA.

SSO Targeting c-Rel Exon 2 Donor Splice Site Efficiently Knockdown c-Rel Expression in B-Lineage Cells

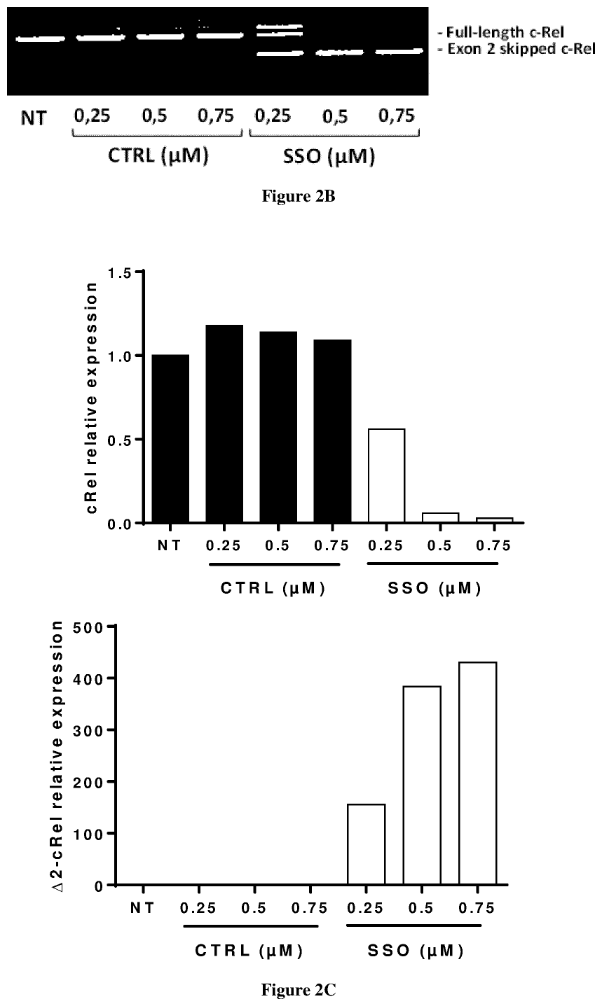

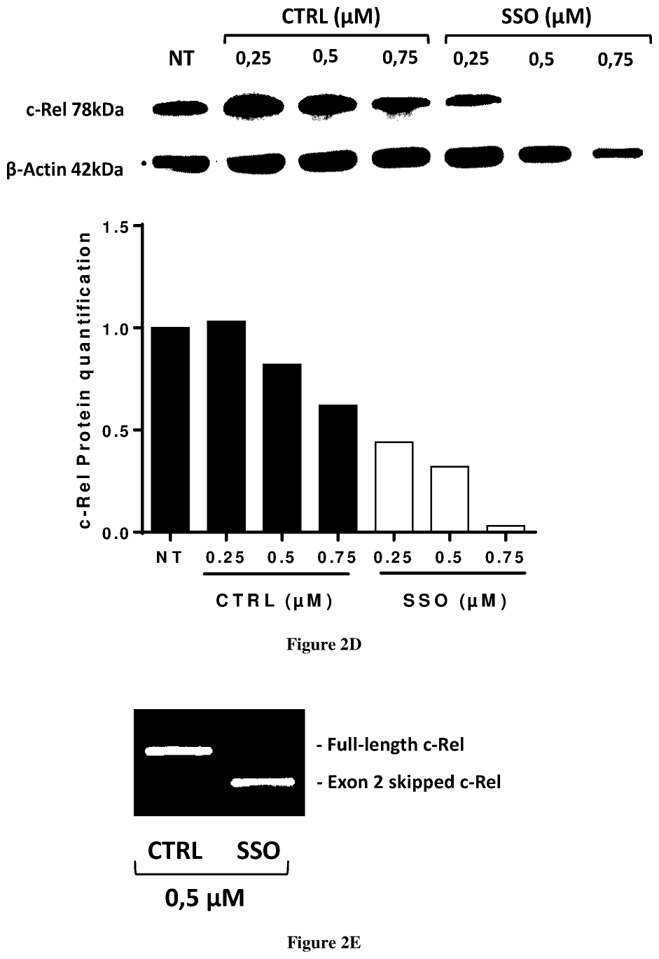

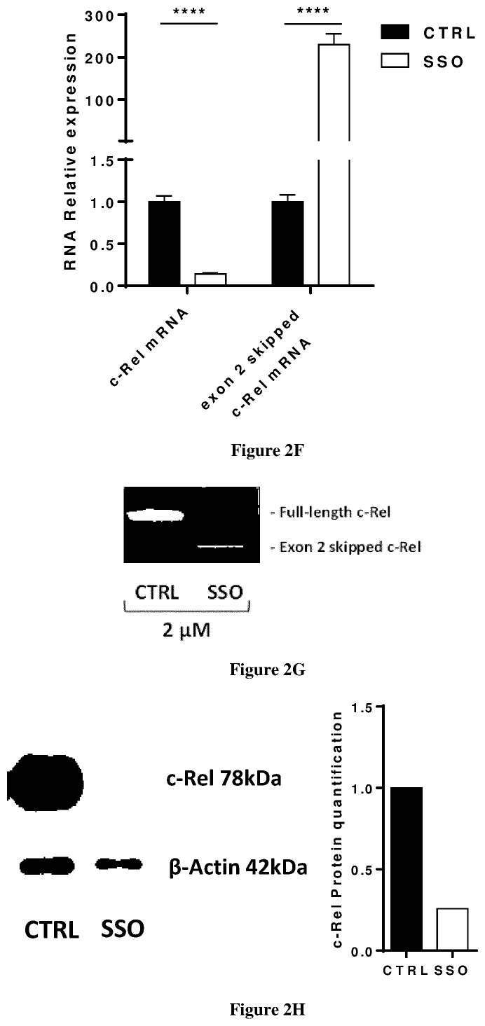

An SSO targeting exon 2 donor splice site of c-Rel was designed with the previously described method in order to convert c-Rel protein translation into an inactive peptide of 21 amino acids, lacking all active domains compared to full-length c-Rel isoform. Consistent with the NMD escape of PTC-containing mRNAs with short ORFs (29,30), alternative c-Rel mRNAs lacking exon 2 are likely poor NMD substrates as the PTC is close to the translation initiation site (29,30) (FIG. 2A). First, SUDHL4 (DLBCL) cells were used to evaluate the efficacy of passive administration of vivo-morpholino SSO targeting c-Rel. Treatments with 0.25 to 0.75 μM vivo-morpholino SSO (SSO) for 48 hours were sufficient to induce a dose-dependent decrease of c-Rel expression, with a 10-fold reduction of c-Rel full-length mRNA expression compared to irrelevant control treated cells (CTRL) (FIG. 2B,C) and a nearly complete absence of c-Rel protein at 0.5 μM dose. As predicted, the c-Rel exon 2 skipped transcript induced in SSO conditions was readily detectable by RT-PCR as it must be poorly degraded by NMD (FIG. 2B). Comparatively, transfection of morpholino SSO was far less efficient than passive administration of vivo-morpholino SSO and did not achieve complete knockdown of c-Rel expression (FIG. 2D). We further verified these results by other experiments on SUDHL4 treated with 0.5 μM of SSO and confirmed an effective reduction of full-length c-Rel mRNA expression (FIG. 2E,F). Then, c-Rel SSO was used in a plasma cell differentiation model described by Le Gallou et al (31,32) and was efficient to provoke c-Rel protein knockdown with a 2 μM dose in pre-plasmablasts (FIG. 2G,H) where c-Rel is still highly expressed and also in plasma cells where its expression is reduced (not shown). Thus, we identified vivo-morpholino SSO targeting c-Rel exon 2 donor splice site as simple and powerful tools to use for the inhibition of c-Rel expression in B and plasma cells.

Treatment with c-Rel Exon 2 Donor Splice Site Targeting SSO Alters Nf-kB Signalling and Induce Apoptosis of DLBCL Cells

SUDHL4 cells treated with 0.5 μM vivo-morpholino SSO were analysed by RNA sequencing to verify the absence of any other in-frame alternative transcripts. Only one out-of-frame junction resulting from intron retention downstream exon 2 was detected in low amount in SSO condition (SSO) but was also present in the irrelevant control condition (CTRL) (not shown). Simulation of translation of this intron retention alternative transcript revealed the presence of a premature stop codon at position 319, making it a good substrate for NMD degradation and unable to restore a partial c-Rel activity. Consistent with the increase of exon2-skipping events (not shown), exon 2 reads including exon1-exon2 and exon2-exon3 junctions decreased while exon1-exon3 junctions appeared when cells were treated with SSO compared to the irrelevant control (CTRL).

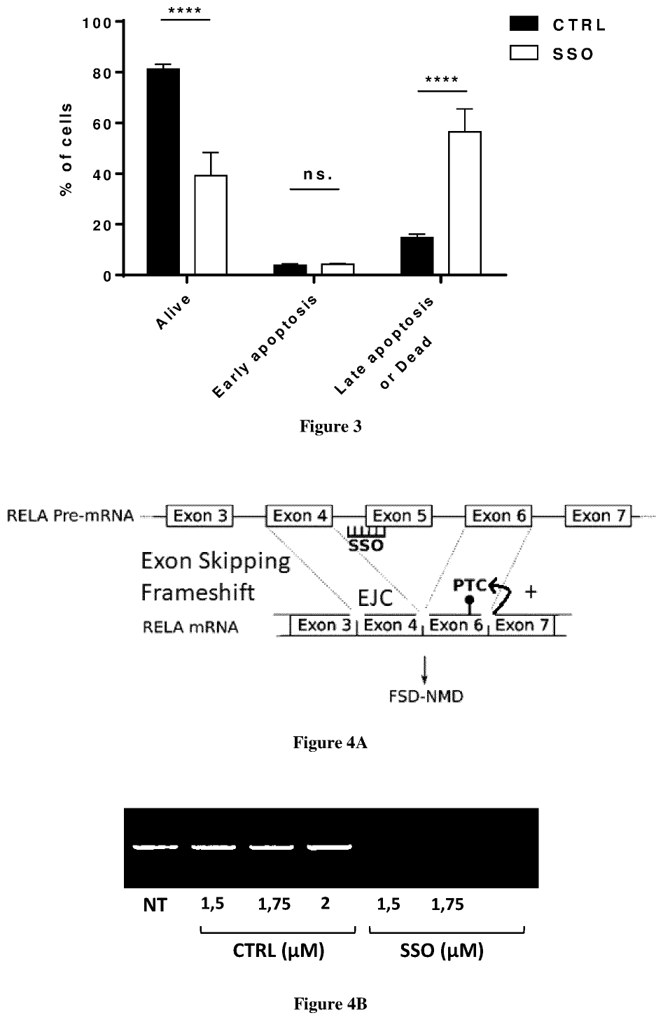

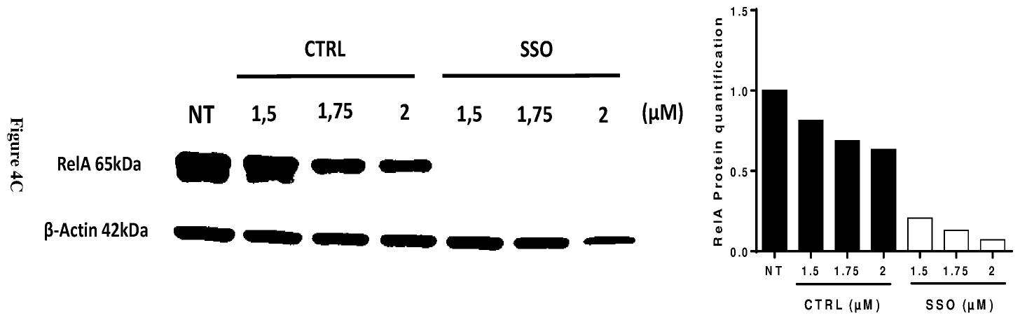

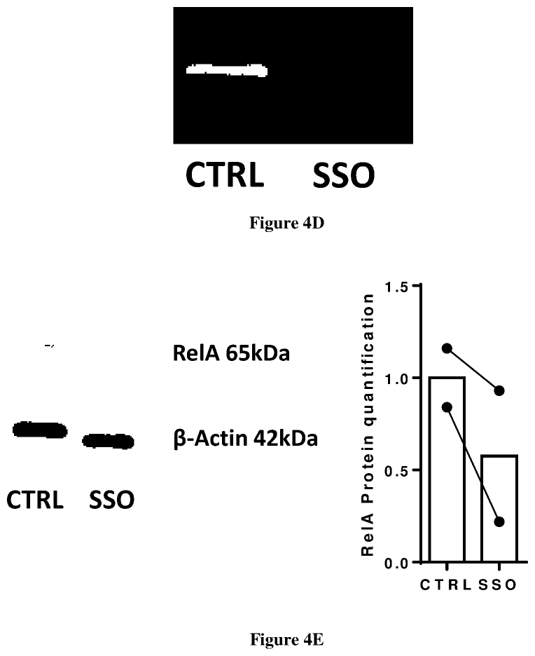

RNA sequencing also revealed that 122 genes were differentially and significantly expressed in DLBCL cells treated with c-Rel SSO compared to control conditions (not shown). Further pathways and gene enrichment analysis revealed that the main differentially expressed pathways were related to NF-κB signalling pathway and immune response to pathogens (FIG. 4C). Among those genes, BCL3 is reduced by almost 2 fold, RELB by 2 fold, NF-κB Inhibitor alpha (IκBα), delta (IκBδ/p100) and zeta (IκBζ) by 1.5, 1.5 and 3 fold respectively, NF-κB2 by 1.5 fold, TNFAIP3/A20, NOTCH2, CD40 and CD83 by more than 1.5 fold and BIRC3 by more than 2.5 fold while PTPN6, VNN1 and FOS increase by 1.5 fold and CNTF increase by 2 fold (not shown). Interestingly, these major transcriptomic modifications, including a deregulated TWEAK (TNF related weak inducer of apoptosis) signaling pathway, were associated with a strong increase of cell death and late apoptosis (FIG. 3).

### RelA Exon 5 Acceptor Splice Site Targeting SSO Efficiently Knockdown RelA Protein in B Cell and Plasmablasts

Another SSO targeting RelA at the exon 5 acceptor splice site was designed with the help of “Exon skipper pipeline” in order to reduce RelA expression. In that case, alternative mRNAs lacking exon 5 are predicted to be NMD targets, with a PTC located at position 460 eliciting interactions with downstream exon junction complex (EJC) components (FIG. 4A). OCILY10 (DLBCL) cells was used to evaluate the efficacy of passive administration of vivo-morpholino SSO to inhibit RelA expression. We observed efficient dose-dependent knockdown of RelA expression with increasing doses of vivo-morpholino SSO (SSO) from 1.5 to 2 μM compared to control conditions (FIG. 4B,C). As expected, full-length RelA mRNAs drastically decreased after SSO treatment, and PTC-containing alternative mRNAs were not detected and likely eliminated by NMD (FIG. 4B). At the protein level, treatment with 2 μM SSO during 48 hours achieved a nearly complete absence of RELA proteins (FIG. 4C). In addition, RelA SSO was tested at 2 μM in a plasma cell differentiation model described by Le Gallou et al (31,32) and compared to irrelevant control SSO (FIG. 4D,E). Again, treatment with RelA SSO diminished the amounts of full-length RelA mRNAs (FIG. 4D) and proteins (FIG. 4E) in plasma cells expressing RelA during their differentiation program.

### Discussion

An efficient transient knockdown of two differentially expressed genes during plasma cell differentiation, c-Rel and RelA, was successfully achieved with an SSO-mediated exon skipping strategy and could constitute a great alternative to RNAi technology with known transfection efficiency limitations in primary B cells and plasma cells (10). In addition, the use of SSO could reduce off-target effects observed with RNAi technology or even RNAse-H1 dependant SSO technologies, as they require direct mRNA degradation actors: RISC/Ago2 complex and RNAse-H1 respectively. Indeed, it was shown that as little as 7 to 11 nucleotides homology between an siRNA and an mRNA was sufficient to recruit RISC/Ago2 complex and induce RNA degradation, particularly in 5′ untranslated regions (33,34). This aspect keeps being a major issue for the development of RNAi-based therapies despite many findings in the field to reduce off-target effects using chemical modifications and optimization of RNAi structure (35). Gapmer ASOs can also recruit RNAse-H1 with unintended target binding and provoke off-target RNA degradation (36). By contrast, SSOs do not directly recruit an RNA degradation machinery and therefore greatly minimize off-target effects. Indeed, SSOs are also capable of weak binding to unintended sequences but the absence of RNA degradation limits the impact on off-target gene expression. However, SSO are not completely deprived of off-target effects as splicing or RNA binding proteins attachment on unintended transcripts can still be disturbed with an SSO (37).

SSO mediated c-Rel inhibition also showed interesting results in a DLBCL cell line, as it was able to reduce NF-κB signalling and cell viability. Interestingly, BCL3 which expression was reduced upon SSO treatment was described to increase cell proliferation by inducing cyclin D1 transcription (38,39). BCL3 is also known to function as an NF-κB inhibitor in the same manner as IκBζ, a Bcl-3 homolog also reduced after SSO treatment, that was shown to potentiate the NF-κB pathway activity by binding to p50 homodimers where they may function as co-activators (40-42). Indeed, Bcl-3 overexpression is found in chronic lymphocytic leukemia where it is frequently rearranged inside IGH locus (43). Additionally, recent findings suggest IκBζ could be a key actor in the development of psoriasis through activation of Th17 immune response (44,45) and could drive the pathogenesis of many haematological cancers (46,47). However, c-Rel is not known to have a direct effect on Bcl-3 and IκBζ expression, the latter preferentially interacting with p50 and p52 (48). Nonetheless, NFKB2 gene encoding p52 which is also reduced upon c-Rel targeting SSO treatment could have influenced Bcl-3 expression as it is tightly regulated by this gene. Accordingly to our results, c-Rel was already found as a direct regulator of NF-κB2 expression in transitional B-cells (49) and could have by this way another indirect effect on RelB expression as NF-κB2 processed protein p52 mainly associate with RelB in the alternative NF-κB pathway (50). Thus, c-Rel inhibition had a broad effect on NF-κB signalling, explaining the decreased viability found upon SSO treatment in DLBCL cells whose tumorigenicity rely on this pathway (51,52). These results still needs to be confirmed in other DLBCL cells lines and patients cells to verify c-Rel knockdown benefits in this pathology but could constitute an interesting lead for new therapy development.

Indeed, targeting NF-κB pathway could be an adequate strategy for the treatment of B-cell malignancies (53) and all NF-κB subunits RelA, c-Rel, RelB, p52 and p50 as well as other NF-κB pathway components such as IKKs or NEMO could be targeted by our SSO induced gene knockdown therapy. Some existing drugs have in fact been found to be active NF-κB inhibitors such as bortezomib which blocks I-κB degradation (54) but no specific NF-κB inhibitor has currently shown promising results in vivo and display an high risk of toxicity (55,56).

To conclude, our results show that an efficient transient gene knockdown can be made by SSO mediated exon skipping with passive administration of vivo-morpholino oligos in primary B cells and plasma cells. Despite some limited exceptions identified with Exon skipper pipeline in mice and humans, this strategy can be applied to most eukaryotic genes and benefit from its transfection-free system in primary cell cultures. Additionally, splicing modulation has the advantage of a low risk of off-target effects, as it does not rely on direct mRNA degradation actors' recruitment. This tool could be an efficient complement to DNA modifications tools such as CRISPR-Cas9 to study normal human B cells and uncover new plasma cell differentiation key mechanisms.

### REFERENCES

Throughout this application, various references describe the state of the art to which this invention pertains. The disclosures of these references are hereby incorporated by reference into the present disclosure.
1. Nutt S L, Hodgkin P D, Tarlinton D M, Corcoran L M. The generation of antibody-secreting plasma cells. Nature Reviews Immunology. 2015 March; 15(3):160-71.
2. Wilmore J R, Allman D. Here, There, and Anywhere? Arguments for and against the Physical Plasma Cell Survival Niche. The Journal of Immunology. 2017 Aug. 1; 199(3):839-45.
3. Suan D, Sundling C, Brink R. Plasma cell and memory B cell differentiation from the germinal center. Current Opinion in Immunology. 2017 Apr. 1; 45:97-102.
4. Khodadadi L, Cheng Q, Radbruch A, Hiepe F. The Maintenance of Memory Plasma Cells. Front Immunol [Internet]. 2019 [cited 2020 Aug. 13]; 10. Available from: https://www.frontiersin.org/articles/10.3389/fimmu.2019.00721/full
5. Lightman S M, Utley A, Lee K P. Survival of Long-Lived Plasma Cells (LLPC): Piecing Together the Puzzle. Front Immunol [Internet]. 2019 [cited 2020 Aug. 13]; 10. Available from: https://www.frontiersin.org/articles/10.3389/fimmu.2019.00965/full
6. Brynjolfsson S F, Persson Berg L, Olsen Ekerhult T, Rimkute I, Wick M-J, Mårtensson I-L, et al. Long-Lived Plasma Cells in Mice and Men. Front Immunol [Internet]. 2018 [cited 2020 Aug. 13]; 9. Available from: https://www.frontiersin.org/articles/10.3389/fimmu.2018.02673/full
7. Libouban H. The use of animal models in multiple myeloma. Morphologie. 2015 Jun. 1; 99(325):63-72.
8. Gado K, Silva S, Paloczi K, Domjan G, Falus A. Mouse plasmacytoma: an experimental model of human multiple myeloma. Haematologica. 2001 Jan. 1; 86(3):227-36.
9. Braggio E, Philipsborn C, Novak A, Hodge L, Ansell S, Fonseca R. Molecular pathogenesis of Waldenström's macroglobulinemia. Haematologica. 2012 Sep. 1; 97(9):1281-90.
10. Shih T, De S, Barnes B J. RNAi Transfection Optimized in Primary Naïve B Cells for the Targeted Analysis of Human Plasma Cell Differentiation. Front Immunol [Internet]. 2019 Jul. 23 [cited 2020 Aug. 13]; 10. Available from: https://www.ncbi.nlm.nih.gov/pmc/articles/PMC6664017/
11. Hung K L, Meitlis I, Hale M, Chen C-Y, Singh S, Jackson S W, et al. Engineering Protein-Secreting Plasma Cells by Homology-Directed Repair in Primary Human B Cells. Molecular Therapy. 2018 Feb. 7; 26(2):456-67.
12. Johnson M J, Laoharawee K, Lahr W S, Webber B R, Moriarity B S. Engineering of Primary Human B cells with CRISPR/Cas9 Targeted Nuclease. Scientific Reports. 2018 Aug. 14; 8(1):12144.
13. Grünewald J, Zhou R, Garcia S P, Iyer S, Lareau C A, Aryee M J, et al. Transcriptome-wide off-target RNA editing induced by CRISPR-guided DNA base editors. Nature. 2019 May; 569(7756):433-7.
14. Fu Y, Foden J A, Khayter C, Maeder M L, Reyon D, Joung J K, et al. High-frequency off-target mutagenesis induced by CRISPR-Cas nucleases in human cells. Nature Biotechnology. 2013 September; 31(9):822-6.
15. Bennett C F. Therapeutic Antisense Oligonucleotides Are Coming of Age. Annual Review of Medicine. 2019; 70(1):307-21.
16. Smith C I E, Zain R. Therapeutic Oligonucleotides: State of the Art. Annual Review of Pharmacology and Toxicology. 2019; 59(1):605-30.
17. Zammarchi F, Stanchina E de, Bournazou E, Supakorndej T, Martires K, Riedel E, et al. Antitumorigenic potential of STAT3 alternative splicing modulation. PNAS. 2011 Oct. 25; 108(43): 17779-84.
18. Wang E T, Sandberg R, Luo S, Khrebtukova I, Zhang L, Mayr C, et al. Alternative isoform regulation in human tissue transcriptomes. Nature. 2008 November; 456(7221):470-6.
19. Maruyama R, Yokota T. Tips to Design Effective Splice-Switching Antisense Oligonucleotides for Exon Skipping and Exon Inclusion. In: Yokota T, Maruyama R, editors. Exon Skipping and Inclusion Therapies: Methods and Protocols [Internet]. New York, NY: Springer; 2018 [cited 2020 Jul. 28]. p. 79-90. (Methods in Molecular Biology). Available from: https://doi.org/10.1007/978-1-4939-8651-4_5
20. Godfrey C, Desviat L R, Smedsrød B, Piétri-Rouxel F, Denti M A, Disterer P, et al. Delivery is key: lessons learnt from developing splice-switching antisense therapies. EMBO Mol Med. 2017 May; 9(5):545-57.
21. Diehl S A, Schmidlin H, Nagasawa M, Haren S D van, Kwakkenbos M J, Yasuda E, et al. STAT3-Mediated Up-Regulation of BLIMP1 Is Coordinated with BCL6 Down-Regulation to Control Human Plasma Cell Differentiation. The Journal of Immunology. 2008 Apr. 1; 180(7):4805-15.
22. Roy K, Mitchell S, Liu Y, Ohta S, Lin Y, Metzig M O, et al. A regulatory circuit controlling the dynamics of the NFκB cRel phases B cells from proliferation to plasma cell differentiation. Immunity. 2019 Mar. 19; 50(3):616-628.e6.
23. Hunter J E, Leslie J, Perkins N D. c-Rel and its many roles in cancer: an old story with new twists. British Journal of Cancer. 2016 January; 114(1):1-6.
24. DiDonato J A, Mercurio F, Karin M. NF-κB and the link between inflammation and cancer. Immunological Reviews. 2012; 246(1):379-400.
25. Raudvere U, Kolberg L, Kuzmin I, Arak T, Adler P, Peterson H, et al. g:Profiler: a web server for functional enrichment analysis and conversions of gene lists (2019 update). Nucleic Acids Res. 2019 Jul. 2; 47(W1):W191-8.
26. Desmet F-O, Hamroun D, Lalande M, Collod-Béroud G, Claustres M, Béroud C. Human Splicing Finder: an online bioinformatics tool to predict splicing signals. Nucleic Acids Res. 2009 May; 37(9):e67.
27. Cartegni L, Wang J, Zhu Z, Zhang M Q, Krainer A R. ESEfinder: a web resource to identify exonic splicing enhancers. Nucleic Acids Res. 2003 Jul. 1; 31(13):3568-71.
28. Zuker M. Mfold web server for nucleic acid folding and hybridization prediction. Nucleic Acids Res. 2003 Jul. 1; 31(13):3406-15.
29. Inácio Â, Silva A L, Pinto J, Ji X, Morgado A, Almeida F, et al. Nonsense Mutations in Close Proximity to the Initiation Codon Fail to Trigger Full Nonsense-mediated mRNA Decay. J Biol Chem. 2004 Jul. 30; 279(31):32170-80.
30. Rebbapragada I, Lykke-Andersen J. Execution of nonsense-mediated mRNA decay: what defines a substrate? Current Opinion in Cell Biology. 2009 Jun. 1; 21(3):394-402.
31. Le Gallou S, Caron G, Delaloy C, Rossille D, Tarte K, Fest T. IL-2 requirement for human plasma cell generation: coupling differentiation and proliferation by enhancing MAPK-ERK signaling. J Immunol. 2012 Jul. 1; 189(1):161-73.
32. Caron G, Hussein M, Kulis M, Delaloy C, Chatonnet F, Pignarre A, et al. Cell-Cycle-Dependent Reconfiguration of the DNA Methylome during Terminal Differentiation of Human B Cells into Plasma Cells. Cell Reports. 2015 Nov. 3; 13(5):1059-71.
33. Lin X, Ruan X, Anderson M G, McDowell J A, Kroeger P E, Fesik S W, et al. siRNA-mediated off-target gene silencing triggered by a 7 nt complementation. Nucleic Acids Res. 2005; 33(14):4527-35.
34. Jackson A L, Bartz S R, Schelter J, Kobayashi S V, Burchard J, Mao M, et al. Expression profiling reveals off-target gene regulation by RNAi. Nature Biotechnology. 2003 June; 21(6):635-7.
35. Suter S R, Sheu-Gruttadauria J, Schirle N T, Valenzuela R, Ball-Jones A A, Onizuka K, et al. Structure-Guided Control of siRNA Off-Target Effects. J Am Chem Soc. 2016 Jul. 20; 138(28):8667-9.
36. Hagedorn P H, Pontoppidan M, Bisgaard T S, Berrera M, Dieckmann A, Ebeling M, et al. Identifying and avoiding off-target effects of RNase H-dependent antisense oligonucleotides in mice. Nucleic Acids Res. 2018 Jun. 20; 46(11):5366-80.
37. Scharner J, Ma W K, Zhang Q, Lin K-T, Rigo F, Bennett CF, et al. Hybridization-mediated off-target effects of splice-switching antisense oligonucleotides. Nucleic Acids Res. 2020 Jan. 24; 48(2):802-16.
38. Park S G, Chung C, Kang H, Kim J-Y, Jung G. Up-regulation of Cyclin D1 by HBx Is Mediated by NF-κB2/BCL3 Complex through κB Site of Cyclin D1 Promoter. J Biol Chem. 2006 Oct. 20; 281(42):31770-7.
39. Westerheide S D, Mayo M W, Anest V, Hanson J L, Baldwin A S. The Putative Oncoprotein Bcl-3 Induces Cyclin D1 To Stimulate G1 Transition. Molecular and Cellular Biology. 2001 Dec. 15; 21(24):8428-36.
40. Yamamoto M, Yamazaki S, Uematsu S, Sato S, Hemmi H, Hoshino K, et al. Regulation of Toll/IL-1-receptor-mediated gene expression by the inducible nuclear protein IκBζ. Nature. 2004 July; 430(6996):218-22.
41. Bours V, Franzoso G, Azarenko V, Park S, Kanno T, Brown K, et al. The oncoprotein Bcl-3 directly transactivates through κB motifs via association with DNA-binding p50B homodimers. Cell. 1993 Mar. 12; 72(5):729-39.
42. Caamaño J H, Perez P, Lira S A, Bravo R. Constitutive expression of Bcl-3 in thymocytes increases the DNA binding of NF-kappaB1 (p50) homodimers in vivo. Mol Cell Biol. 1996 April; 16(4):1342-8.
43. McKeithan T W, Takimoto G S, Ohno H, Bjorling V S, Morgan R, Hecht B K, et al. BCL3 rearrangements and t(14;19) in chronic lymphocytic leukemia and other B-cell malignancies: A molecular and cytogenetic study. Genes, Chromosomes and Cancer. 1997; 20(1):64-72.
44. Müller A, Hennig A, Lorscheid S, Grondona P, Schulze-Osthoff K, Hailfinger S, et al. IκBζ is a key transcriptional regulator of IL-36-driven psoriasis-related gene expression in keratinocytes. Proc Natl Acad Sci USA. 2018 Oct. 2; 115(40):10088-93.
45. Okamoto K, Iwai Y, Oh-Hora M, Yamamoto M, Morio T, Aoki K, et al. IkappaBzeta regulates T(H)17 development by cooperating with ROR nuclear receptors. Nature. 2010 Apr. 29; 464(7293): 1381-5.
46. Willems M, Dubois N, Musumeci L, Bours V, Robe P A. IκBζ: an emerging player in cancer. Oncotarget. 2016 Aug. 26; 7(40):66310-22.
47. Nogai H, Wenzel S-S, Hailfinger S, Grau M, Kaergel E, Seitz V, et al. IκB-ζ controls the constitutive NF-κB target gene network and survival of ABC DLBCL. Blood. 2013 Sep. 26; 122(13):2242-50.
48. Nolan G P, Fujita T, Bhatia K, Huppi C, Liou H C, Scott M L, et al. The bcl-3 proto-oncogene encodes a nuclear I kappa B-like molecule that preferentially interacts with NF-kappa B p50 and p52 in a phosphorylation-dependent manner. Mol Cell Biol. 1993 June; 13(6):3557-66.
49. Castro I, Wright J A, Damdinsuren B, Hoek K L, Carlesso G, Shinners N P, et al. B Cell Receptor-Mediated Sustained c-Rel Activation Facilitates Late Transitional B Cell Survival through Control of B Cell Activating Factor Receptor and NF-κB2. The Journal of Immunology. 2009 Jun. 15; 182(12):7729-37.
50. Kawai T, Akira S. Signaling to NF-κB by Toll-like receptors. Trends in Molecular Medicine. 2007 Nov. 1; 13(11):460-9.
51. Nogai H, Dörken B, Lenz G. Pathogenesis of Non-Hodgkin's Lymphoma. JCO. 2011 Apr. 11; 29(14):1803-11.
52. Young R M, Shaffer A L, Phelan J D, Staudt L M. B-Cell Receptor Signaling in Diffuse Large B-Cell lymphoma. Seminars in Hematology. 2015 Apr. 1; 52(2):77-85.
53. Perkins N D. The diverse and complex roles of NF-κB subunits in cancer. Nature Reviews Cancer. 2012 February; 12(2):121-32.
54. Roccaro A M, Ribatti A V and D. Bortezomib in the Treatment of Cancer [Internet]. Vol. 1, Recent Patents on Anti-Cancer Drug Discovery. 2006 [cited 2020 Aug. 26]. p. 397-403. Available from: https://www.eurekaselect.com/77311/article
55. Perkins N D. Integrating cell-signalling pathways with NF-κB and IKK function. Nature Reviews Molecular Cell Biology. 2007 January; 8(1):49-62.
56. Chariot A. The NF-κB-independent functions of IKK subunits in immunity and cancer. Trends in Cell Biology. 2009 Aug. 1; 19(8):404-13.

1. A method of reducing expression of a NF-κB component in B cells of a subject in need thereof comprising administering to the subject an effective amount of at least one splice switching antisense oligonucleotide targeting a splice site of one exon, or a splicing regulatory sequence, in the pre-mRNA molecule encoding the NF-κB component to alter splicing by blocking recognition of said splice site by splicing machinery and thus inducing exon skipping and reducing expression of the NF-κB component.

2. The method of claim 1 wherein the at least one splice switching antisense oligonucleotide (SSO) mediates the exon-skipping for a pre-mRNA having at least 3 exons, wherein a targeted internal exon has a number of nucleotides not divisible by 3, thereby inducing a reading frameshift.

3. The method of claim 1 wherein the at least one splice switching antisense oligonucleotide is an antisense RNA or DNA.

4. The method of claim 1, the expression of c-REL in the B cells of the subject is reduced.

5. The method of claim 4 wherein the at least one splice switching antisense oligonucleotide targets the c-REL exon 2 donor splice site.

6. The method of claim 4 wherein the at least one splice switching antisense oligonucleotide is complementary to the nucleic acid sequence as set forth in SEQ ID NO:5.

7. The method of claim 4 wherein the at least one splice switching antisense oligonucleotide converts c-Rel protein translation into an inactive peptide of 21 amino acids, lacking all active domains compared to full-length c-Rel isoform and having the amino acid sequence as set forth in SEQ ID NO:6.

8. The method of claim 4 wherein the at least one splice switching antisense oligonucleotide targets the c-REL exon 2 donor splice site and comprises the sequences as set forth in SEQ ID NO:7.

9. The method of claim 1, wherein expression of RELA in the B cells of the subject is reduced.

10. The method of claim 9 wherein the at least one splice switching antisense oligonucleotide targets the RELA acceptor splice site of exon 5.

11. The method of claim 9 wherein the at least one splice switching antisense oligonucleotide is complementary to the nucleic acid sequence as set forth in SEQ ID NO:8.

12. The method of claim 9 wherein the at least one splice switching antisense oligonucleotide targets the RELA acceptor splice site of exon 5 and comprises the sequence as set forth in SEQ ID NO:9.

13. The method of claim 1, wherein expression of RELB in the B cells of the subject is reduced.

14. The method of claim 13, wherein the at least one splice switching antisense oligonucleotide targets the RELB acceptor splice site of exon 5 and comprises the sequence as set forth in SEQ ID NO:10.

15. The method of claim 1, wherein expression of IKK2 in the B cells of the subject is reduced.

16. The method of claim 15 wherein the at least one splice switching antisense oligonucleotide targets the IKK2 acceptor splice site of exon 3 and comprises the sequence as set forth in SEQ ID NO:11.

17. The method of claim 15 wherein the at least one splice switching antisense oligonucleotide targets the IKK2 acceptor splice site of exon 5 and comprises the sequence as set forth in SEQ ID NO:12.

18. The method of claim 1 wherein the at least one splice switching antisense oligonucleotide is stabilized.

19. The method of claim 1 wherein the subject suffers from a B cell malignancy.

20. The method of claim 1 wherein the subject suffers from multiple myeloma.

21. The method of claim 1 wherein the subject suffers from a disease associated with autoimmunity or inflammation.

22. A splice switching antisense oligonucleotide that comprises the sequence as set forth in SEQ ID NO:7, SEQ ID NO:9, SEQ ID NO:10, SEQ ID NO:11 or SEQ ID NO:12.

23. The method of claim 19, wherein the B cell malignancy is diffuse large B cell lymphoma.
